(19) United States
(12) Patent Application Publication
(10) Pub. No.: US 2025/0044272 A1
(43) Pub. Date: Feb. 6, 2025

(54) APPARATUS FOR MEASURING WATER QUALITY OF SEWAGE AND CONTROL METHOD THEREOF

(71) Applicant: WATER-EYES CO. LTD., Sejong (KR)

(72) Inventors: Yeon Wuk JUNG, Sejong (KR); Jeong Hun HAN, Daejeon (KR); Ye Jin KIM, Sejong (KR)

(73) Assignee: WATER-EYES CO. LTD., Sejong (KR)

(21) Appl. No.: 18/277,290

(22) PCT Filed: Aug. 1, 2023
(86) PCT No.: PCT/KR2023/011205

§ 371 Date: Aug. 15, 2023

(30) Foreign Application Priority Data
Nov. 23, 2022 (KR) 10-2022-0158736

(51) Int. Cl. G01N 33/18; B08B 3/04

(52) U.S. Cl. CPC G01N 33/18; B08B 3/04

(57) ABSTRACT

An electronic device for measuring water quality of inflow sewage, the electronic device comprising: a sensor part installed inside a pipeline of the inflow sewage and including at least one sensor for measuring a water quality item of the inflow sewage and outputting a sensing value; a communication part for communicating with a server; and a controller for processing and transmitting the sensing value to the server, wherein the sensor part further includes a pollution prevention part for emitting a fluid toward an electrode part of the sensor, and wherein the controller controls the pollution prevention part to emit the fluid at a first predetermined period.

### TECHNICAL FIELD

The disclosed invention relates to a water quality of sewage measuring apparatus and a control method thereof, and particularly, to a water quality of sewage measuring apparatus and a control method thereof capable of providing a warning to a user by measuring water quality even inflowing sewage with high pollution levels and predicting water quality discharged therefrom.

### BACKGROUND ART

Recently, as environmental interests increase, various environmental pollutions have emerged as social problems, and among them, the problem of water pollution is more serious due to the contamination of food water necessary for human survival.

In order to prevent such serious water pollution, the government has established five items of sewage treatment facilities by legal measures: a biological oxygen demand (BOD), a chemical oxygen demand (COD), a suspended solid (SS), a total nitrogen (T-N, total nitrogen), a total phosphorus (T-P), and a total organic carbon (TOC) to manage discharge water quality. Accordingly, if water quality management criteria are not met, warnings and business suspensions will be issued.

However, due to the characteristics of the sewage treatment process using microorganisms, a significant time such as about 1 to 2 weeks is required to normalize the contaminated microorganisms, and when unknown water is introduced, water quality rapidly deteriorates within about 2 hours. When sewage having high pollution degrees is introduced or unknown water not introduced through sewage trench is introduced, the standard of discharge water cannot be satisfied due to the limitation of the characteristics of the sewage treatment facility, and thus an emergency work condition occurs.

In order to solve these problems, it is necessary to previously measure the water quality of sewage introduced into the sewage treatment facility, thereby performing an additional process in advance and satisfying the water quality standard of discharge water of the sewage treatment facility.

Here, as an example of the conventional water quality sensor for measuring the water quality as described above, according to Korean Patent Laid-Open No 10-1261737, there is provided a water quality measurement apparatus including: a case part including a rear case having a predetermined internal space and a front case fastened to the front surface of the rear case; a circuit module seated on a bottom surface of the rear case; a housing electrically connected to the circuit module and including a first reservoir formed to measure the water quality of water and having a predetermined height, a receiving space connected to an inlet hole provided at one side of the outer surface of the second reservoir, and a plurality of flow passages configured to communicate the first reservoir, the second reservoir, and the receiving space with each other; a chamber accommodated in the receiving space and including a plurality of divided spaces communicating with the inlet hole; and a sensing module including a first reservoir, a second reservoir, and a plurality of sensors provided in the plurality of divided spaces to measure different factors of water introduced into the plurality of divided spaces, so that items such as nitrogen concentration, chlorine concentration, hydrogen concentration, and electrical conductivity forming water quality of water degrees are measured in the divided spaces, thereby increasing reliability of multiitem measurement.

Further, as another example of the related art for a water quality sensor for measuring the water quality as described above, according to, for example, Korean Patent No 10-1221646 discloses a water quality monitoring device for a soda hood, which includes: a base plate fixedly installed on a bottom surface of the soda hood; a sensor housing fixedly installed at an eccentric position of the base plate, and having a cross-sectional area of an inlet through which water is introduced, wider than a cross-sectional area of an outlet through which water is discharged; a water quality sensor inserted and installed on one side wall surface of the sensor housing to sense the water quality of water passing through the sensor housing; and a screen unit fixedly installed on a periphery of the base plate to block the inflow of foreign substances other than water through the front, rear, left, right, and upper surfaces of the water quality sensor, so that it is possible to monitor the water quality by installing the base plate at a location where the water depth is low, such as the soda hood, and a water quality monitoring device for a soda hood is configured to improve the safety and durability of the water quality monitoring device by protecting the water quality sensor in duplicate using the screen unit and the sensor housing.

As described above, various technical contents related to the water quality sensor have been suggested, but the related art water quality sensors have the following problems.

### DISCLOSURE

### Technical Problem

An aspect of the disclosed invention provides a water quality of sewage measurement apparatus capable of measuring water quality in inflow sewage with high pollution levels so as to predict water quality measurement items of discharge water and a control method thereof.

### Technical Solution

The electronic device for measuring water quality of sewage according to an aspect of the disclosed invention includes a sensor part installed inside a pipeline of the inflow sewage and including at least one sensor for measuring a water quality item of the inflow sewage and outputting a sensing value, a communication part for communicating with a server, and a controller for processing and transmitting the sensing value to the server, wherein the sensor part further includes a pollution prevention part for emitting a fluid toward an electrode part of the sensor, and the controller may control the pollution prevention part to emit the fluid at every first predetermined period.

The sensor may include a main body extending in a longitudinal direction of a cylindrical shape, at least one electrode part protruding in the longitudinal direction from the main body, and a protection part wherein one end of the protection part is connected to an outer circumference of one end of the main body and extends in the longitudinal direction to surround the protruding electrode part, and the other end is opened to have a space provided therein.

The pollution prevention part may include a fluid passage, wherein one end of the fluid passage is connected to a pump part so that a fluid may move through the main body, and wherein the protection part may include at least one hole, wherein the at least one hole is provided toward the electrode part inside the protection part and connected to the fluid passage.

At least a portion of an outer circumferential surface of the protection part is opened so that foreign substances removed by the pollution prevention part move.

The sensor may further include a connection part capable of transmitting and receiving the sensing value, and the electronic device may further include a connection unit for detachably coupling the connection part.

The sensor part may include a first sensor and a second sensor, and the controller may control the pollution prevention part to emit the fluid when a change amount of at least one sensing value greater than a predetermined value is identified during a second predetermined period based on processing of the sensing values of the first sensor and the second sensor.

The sensor may include at least one DO sensor, a pH sensor, an electrical conductivity sensor, a mixed liqueur suspended solid (MLSS) sensor, a turbidity sensor, or a residual chlorine sensor.

The method for measuring water quality of sewage according to an aspect of the disclosed invention may include measuring a water quality item of the inflow sewage by a sensor part including at least one sensor and outputting at least one sensing value, processing the sensing value by the controller and transmitting the processed sensing value to the server through a communication part, and controlling the pollution prevention part to emit the fluid toward an electrode of the sensor part at every first predetermined period.

### Advantageous Effects

According to an aspect of the disclosed invention, it is possible to provide a water quality of sewage measurement apparatus capable of performing water quality measurement in inflow sewage with high pollution level and a control method thereof.

### DESCRIPTION OF DRAWINGS

FIG. 1 is a conceptual diagram for explaining a water quality of sewage measurement system according to an embodiment.

FIG. 2 is a block diagram illustrating a configuration of a water quality of sewage measurement electronic device according to an embodiment.

FIG. 3 is a flowchart for explaining a water quality of sewage measurement method according to an embodiment.

FIG. 4 is a flowchart for explaining a water quality of sewage measurement method according to an embodiment.

### MODES OF THE INVENTION

Throughout the specification, the same reference numerals refer to the same components. The present specification does not describe all elements of the embodiments, and general contents or duplicate contents between the embodiments in the art to which the disclosed invention pertains will be omitted. The terms “unit, module, member, and block” used in the specification may be implemented by software or hardware, and a plurality of “units”, “modules, members, and blocks” may be implemented by one component or one “unit, module, member, and blocks” may include a plurality of components according to embodiments.

Throughout the specification, when a part is referred to as being “connected” to another part, the part may include not only a case where the part is directly connected but also a case where the part is indirectly connected, and the indirect connection may include a case where the part is connected through a wireless communication network.

In addition, when a part “includes” a component, unless otherwise specified, the part may further include another component, not excluding the other component.

The terms “first”, “second”, and the like are used to distinguish one component from another component, and the components are not limited by the above-described terms.

The singular forms “a”, “an”, and “the” include plural forms unless the context clearly dictates otherwise.

In each step, the identification numerals are used for convenience of description, and the identification numerals do not describe the order of each step, and each step may be performed in a different manner from the specified order unless the context clearly dictates a specific order.

Hereinafter, the working principles and embodiments of the disclosed invention will be described with reference to the accompanying drawings.

The present invention may be directed to solve a problem in that it is difficult to measure and analyze water quality of sewage introduced by a method of measuring and analyzing water quality only to be installed at a final origin of a sewage treatment facility due to a large limitation on the size, structure, installation cost, installation method and procedure of a water quality measuring apparatus and the durability of sophisticated sensors based on high pollution of inflow sewage.

More specifically, a general water quality analysis method of inflow sewage takes about 6 hours to about 8 hours, and a corresponding strength of workers to sewage treatment processes increases due to a significant time consumption. In addition, sewage having high pollution has a problem in that the durability of sophisticated digital sensors rapidly decreases and costs increases.

That is, the water quality specific of the inflow sewage using a sophisticated sensor that directly measures the biological oxygen demand (BOD), the chemical oxygen demand (COD), the suspended solid (SS), the total nitrogen (T-N, Total Nitrogen), the total phosphorus (T-P), and the total organic carbon (TOC), which are the water quality measurement items, is rapidly reduced durability, and thus it is necessary to replace the inflow sewage every fast cycles, and there is a problem that costs occur and water quality of the inflow sewage cannot be continuously monitored.

To solve the problem, the present invention provides a water quality of sewage measurement electronic device capable of measuring and providing a water quality basic item (e.g., residual chlorine, dissolved oxygen (DO), oxidity (pH), electrical conductivity, turbidity type, and mixed liquor suspended solid sensor (MLSS)), rather than a sensor having a fast replacement cycle, in consideration of the characteristics of the inflow sewage having high pollution. Accordingly, the electronic device of the present invention transmits the water quality basic items to a server, thereby predicting the water quality measurement item using a model predicting the water quality measurement item of the server. Accordingly, the server provides the predicted water quality measurement item to the user terminal, thereby easily grasping the pollution of the inflow sewage and the possibility of occurrence of a problem.

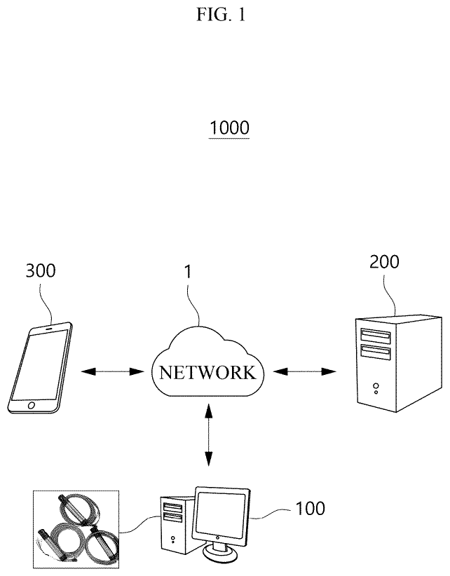

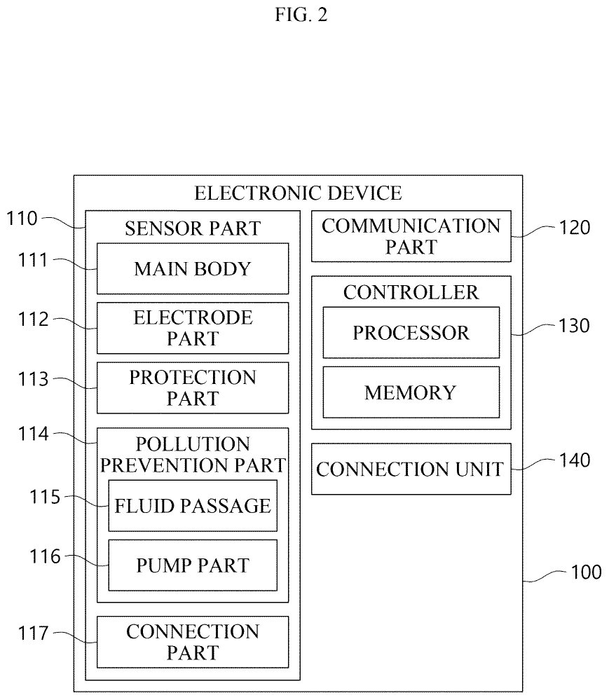

FIG. 1 is a conceptual diagram for describing a water quality of sewage measurement system 1000 according to an embodiment. FIG. 2 is a block diagram illustrating a configuration of a water quality of sewage measurement electronic device according to an embodiment.

As illustrated in FIG. 1, the water quality of sewage measurement system 1000 according to an embodiment of the present invention may include a sewage measurement electronic device 100 capable of measuring basic water quality measurement items of inflow sewage and transmitting the measured basic water quality measurement items to a server 200, and cleaning a contaminated sensor by itself, a server 200 for predicting the water quality measurement item by inputting the basic water quality measurement items received from the water quality of sewage measurement electronic device 100 to a water quality measurement item prediction model and outputting the water quality measurement item, and a user terminal 300 for displaying the predicted water quality measurement item and the basic water quality measurement item.

More specifically, the basic water quality measurement item according to the embodiment of the present invention may include, for example, the water quality basic item (e.g., residual chlorine, dissolved oxygen (DO), oxidity (pH), electrical conductivity, turbidity type, and mixed liquor suspended solid sensor (MLSS), but is not limited thereto. In addition, the reference water quality measurement item according to the embodiment of the present invention may include, for example, the biological oxygen demand (BOD), the chemical oxygen demand (COD), the suspended solid (SS), the total nitrogen (T-N, Total Nitrogen), the total phosphorus (T-P), and the total organic carbon (TOC), and the like. However, the present invention is not limited thereto. In particular, it may be understood that the above-described basic water quality measurement item and the reference water quality measurement item may be added according to the utilization of an additional sensor or the addition of a future government's reference water quality measurement item.

The water quality of sewage measurement system 1000 according to an embodiment of the present invention may be connected to the water quality of sewage measurement electronic device 100, the server 200, and the user terminal 300. Here, it may be understood that the meaning that the network 1 may be connected may be electrically or communicatively connected.

Examples of the network 1 include, but are not limited to, a 3rd Generation Partnership Project (3GPP) network, a Long Term Evolution (LTE) network, a 5G network, a World Interoperability for Microwave Access (WMAX) network, an Internet, a Local Area Network (LAN), a Wireless LAN (WLAN), a Wide Area Network (WAN), a Personal Area Network (PAN), a Bluetooth network, a satellite broadcast network, an analog broadcast network, a Digital Multimedia Broadcasting (DMB) network, and the like.

The water quality of sewage measurement electronic device 100 according to an embodiment of the present invention may measure both water quality of a water supply and a sewage only by replacing the sensor. More specifically, as will be described in detail below, the water quality of sewage measurement electronic device 100 includes a connection unit that is detachable from the sensor, and thus it may be understood that the residual chlorine sensor necessary for measuring the water quality of the water supply may be detachable, and accordingly, the water quality of sewage measurement electronic device 100 may be suitably applied to measuring the water quality of the water supply.

The water quality of sewage measurement electronic device 100 according to an embodiment of the present invention may transmit a sensing value to the server 200 based on processing of a sensing value measured from a sensor measured inside a pipeline of inflow sewage. In particular, the water quality of sewage measurement device 100 may emit a fluid for washing the sensor at a predetermined period, and thus may wash contaminants such as suspended substances to derive an accurate sensing value of the sensor.

In addition, when the water quality of sewage measurement electronic device 100 includes a plurality of sensors, the water quality of sewage measurement electronic device 100 may identify a change amount of at least one sensing value greater than a predetermined value during a predetermined period based on processing of the plurality of sensing values. Accordingly, the water quality of sewage measurement electronic device 100 may control the emit of the fluid for washing the sensor. Since the plurality of sensing values have a correlation, the plurality of sensing values may identify a case where there is no change in another sensing value even when one sensing value exhibits a change amount greater than the predetermined value, and thus may control the corresponding sensor to be washed. However, the present invention is not limited thereto.

The server 200 according to an embodiment of the present invention may process and store various kinds of data and transmit and/or receive the processed data. More specifically, the server 200 may be connected to the water quality of sewage measurement electronic device 100 and the user terminal 300 with each other as the network 1. Accordingly, the server 200 may transmit and/or receive data for interworking data with each other based on the network 1. The server 200 may be, for example, a platform, but is not limited thereto.

The server 200 may receive a request for prediction For example, the server 200 may transmit the basic water quality measurement item received from the water quality of sewage measurement electronic device 100 other than the reference water quality measurement item to the user terminal 300. In addition, the server 200 may update values for the current basic water quality measurement item in response to receiving the basic water quality measurement item from the water quality of sewage measurement electronic device 100.

The server 200 may be integrated with the database, and may search for corresponding information based on big data stored in the database, and may reconstruct the searched information appropriately and transmit the corresponding information to the user terminal 300 again when a request for specific information (e.g., the basic water quality measurement item and/or the water quality measurement item) is received from each user through the user terminal 300. That is, the server 200 may accumulate all data received from the water quality of sewage measurement electronic device 100 and may store big data by using each information as a database.

The server 200 may include at least one processor for processing various kinds of data. For example, the processor of the server 200 may include a learning processor for machine learning and may train a model including an artificial neural network using learning data. Here, the trained artificial neural network may be referred to as a learning model. The learning model may be used to infer a result value for new input data, not learning data, and the inferred value may be used as a basis for a determination for performing any operation.

More specifically, the processor of the server 200 may analyze and process data based on raw data of the sewage treatment facility (herein, the raw data may be understood as learning data) to build a machine learning model. In order to secure reliability of the reliability, the machine learning model may be built by using water environment information system data as additional raw data. The machine learning model thus built may be referred to as a reference water quality measurement item prediction model, for example. However, the present invention is not limited thereto.

Here, the raw data and the water environment information system data of the sewage treatment facility may be machine learning through supervised learning, for example, based on the basic water quality measurement item of the inflow sewage and the actually measured reference water quality measurement item. More specifically, the correlation between the basic water quality measurement item and the reference water quality measurement item may be trained such that the reference water quality measurement item is output as an input of the basic water quality measurement item of the inflow sewage. However, the present invention is not limited thereto.

In another embodiment, the processor of the server 200 may train the learning model through a deep learning algorithm.

The deep learning algorithm refers to a modeling technique developed in an artificial neural network taken from a human neural network as one of machine learning algorithms. The artificial neural network may be configured in a multi-layered hierarchical structure.

The artificial neural network (ANN) may be configured in a hierarchical structure including an input layer, an output layer, and at least one or more intermediate layers (or hidden layers, e.g., kernels) between the input layer and the output layer. The deep learning algorithm may derive a result of a highly reliable result through learning of optimizing a weight of an interlayer activation function based on the multi-hierarchical structure.

The deep learning algorithm applicable to the processor of the server 200 according to an embodiment of the present disclosure may include, for example, a convolutional neural network (CNN). However, the present disclosure is not limited thereto. It is understood that other deep learning algorithms may be applied according to embodiments.

Unlike a technique in which a learning process is performed by extracting knowledge from existing data, the convolutional neural network (CNN) has a structure in which features of data are extracted to determine patterns of features. The convolutional neural network (CNN) may be performed through a convolution process and a pooling process. In other words, the convolutional neural network (CNN) may include an algorithm in which a convolution layer and a pooling layer are configured in a complex manner. Here, a process (e.g., a convolutional process) of extracting features of data is performed in the convolution layer. The convolution process is a process of determining features by examining adjacent components of each component to data and deriving the determined features as a single piece, and the number of parameters may be effectively reduced by one compression process. In the pooling layer, a process (e.g., a pooling process) of reducing the size of a layer that has undergone the convolution process is performed. The pooling process may reduce the size of data, cancel noise, and provide consistent features in a fine part. For example, the convolutional neural network (CNN) may be used in various fields such as information extraction, sentence classification, and face recognition. Meanwhile, since the convolutional neural network (CNN) is a well-known technology, detailed descriptions thereof will be omitted below.

Accordingly, the server 200 may input the basic water quality measurement item received from the water quality of sewage measurement electronic device 100 to the reference water quality measurement item prediction model and output the predicted reference water quality measurement item, thereby predicting the reference water quality measurement item of the inflow sewage with high reliability. Accordingly, the water quality of sewage measurement system may provide information to the user to prepare for an emergency situation based on the degree of pollution of the inflow sewage and the amount of cleaning limits of the sewage treatment facility.

Meanwhile, the server 200 may determine the possibility that water quality items of the discharge water exceed the reference water quality measurement item based on the predicted reference water quality measurement item. Accordingly, when the inflow sewage is highly likely to exceed the reference water quality measurement item, the server 200 may provide an alarm to the user terminal 300. However, the present disclosure is not limited thereto. As another embodiment, it is understood that a new algorithm for determining the possibility of exceeding the reference water quality measurement item may be applied, and the alarm may be provided differently by setting a section for probability.

The user terminal 300 according to an embodiment of the present invention may include, for example, all kinds of input/output cable/wireless communication devices such as a Personal Communication System (PCS), Global System for Mobile communication (GSM), Personal Digital Cellular (PDC), Personal Handyphone System (PHS), Personal Digital Assistant (PDA), International Mobile Telecommunication (IMT)-2000, Code Division Multiple Access (CDMA)-2000, W-Code Division Multiple Access (W-CDMA), Wireless Broadband Internet (Wibro) terminal, a smartphone, a smart pad, a tablet PC, a notebook, a wearable device, a digital signage, and the like, but is not limited thereto.

In addition, the user terminal 300 may include a manager terminal having a difference between accessible data and viewable data. More specifically, the user terminal 300 may request information on a basic water quality measurement item of the current inflow sewage from the server 200. In addition, the user terminal 300 may request information on a reference water quality measurement item predicted based on the basic water quality measurement item of the current inflow sewage from the server 200.

In response to this, the server 200 may transmit the requested information to the user terminal 300.

Hereinafter, a configuration of the water quality of sewage measurement electronic device 100 according to an embodiment of the present invention will be described in detail with reference to FIG. 2.

Referring to FIG. 2, the water quality of sewage measurement electronic device 100 may include a sensor part 110, a communication part 120, a controller 130, and a connection unit 140.

The sensor part 110 according to an embodiment of the present invention may include at least one sensor installed inside a pipeline of the inflow sewage to measure a water quality item of the inflow sewage and output a sensing value. Here, the sensor may include at least one DO sensor, a pH sensor, an electrical conductivity sensor, a Mixed Liquor Suspended Solid (MLSS) sensor, a turbidity sensor, or a residual chlorine sensor. However, the present invention is not limited thereto. That is, as described above, it is understood that additional sensors may be utilized, and may be added in accordance with the addition of a government's discharge water reference water quality measurement item and a basic water quality item correlated with the added reference water quality measurement item in the future.

The sensor part 110 may include a main body 111, an electrode part 112, a protection part 113, a pollution prevention part 114, and a connection part 117. Meanwhile, the sensor part 110 may mean including at least one sensor. That is, the sensor part 110 including one sensor may be named the same as the sensor. Hereinafter, the description will be made with reference to the sensor part 110 including one sensor.

The main body 111 of the sensor part 110 may include a cylindrical shape and may extend in a longitudinal direction of the cylindrical shape and. However, the present invention is not limited thereto. It is understood that the main body 111 having various structures may be applied, and for example, the main body 111 may be provided to have an inner space to minimize resistance to the flow of the inflow sewage.

The main body 111 of the sensor part 110 may have a cylindrical shape extending in the longitudinal direction of the cylindrical shape and may have a fluid passage 115 provided therein with a space to allow the fluid emitted by the pollution prevention part 114 of the sensor part 110 to move. In addition, the main body 111 may be connected to the electrode part 112 for measuring the water quality of the inflow sewage and may communicate with the connection part 117 for transmitting the sensing value to the controller 130. That is, the main body 111 may have a shape surrounding the connection part 117 and the fluid passage 115, which are internal data transmission/reception lines of the sensor 110, and may prevent damage to the connection part 117 and the fluid passage 115.

In addition, the sensor part 110 may include at least one electrode part 112 protruding in the longitudinal direction of the main body. That is, the sensor part 110 may be configured as a galvanic sensor having a structure in which only the tip of the electrode part 112 is replaced, and may be configured to facilitate maintenance. However, the structure of the electrode part 112 may be changed according to the type of the sensor included in the sensor part 110, without being limited thereto. That is, the shape of the electrode part 112 may be changed, and accordingly, the sensor item to be measured may be changed. For example, when the electrical conductivity sensor is used, it may be understood that the electrode part 112 may be configured such that at least one pair of electrodes protrude.

The sensor part 110 may include a protection part 113 having one end connected to an outer circumference of one end of the main body 111 and extending in a longitudinal direction to surround the protruded electrode part 112, and the other end opened to have a space provided therein. That is, the protection part 113 of the sensor 110 may have a shape surrounding the electrode part 112 of the sensor 110 and may be open in the longitudinal direction of the main body 111 to move the inflowing sewage.

In more detail, the protection part 113 of the sensor part 110 may have the electrode part 112 disposed therein in the same form as a cup having an empty inside so as to protect the electrode part 112 from a force in a lateral direction perpendicular to the longitudinal direction of the main body 111.

That is, the shape of the main body 111, the electrode part 112, and the protection part 113 of the sensor part 110 may have, for example, a cylindrical shape extending in a longitudinal direction, and the inside of the second section among the first section and the second section in the longitudinal direction may have a empty shape. That is, the protection part may mean a second section. In addition, since the inside of the second section has an empty shape, the electrode part 112 protruding in the longitudinal direction may be disposed inside the inside. However, the present invention is not limited thereto.

Meanwhile, the protection part 113 may include a structure in which at least a part of the outer circumferential surface is opened so that foreign substances removed by the fluid emitted by the pollution prevention part 114 to be described later may move. In particular, when at least a portion of the outer circumferential surface of the protection part 113 is opened, since the flow of inflow sewage is smoother than the protection part 113 opened in the longitudinal direction of the main body 111, more sophisticated sensing may be enabled.

More specifically, at least a part of the outer circumferential surface of the protection part 113 is opened may mean that the protection part 113 extending from the main body 111 is empty inside and has a shape in which one end is connected to the main body 111 and the other end is opened, and thus at least a part of the outer circumferential surface of the protection part 113 may include a hole (no shape limitation) to allow the fluid to move in a direction perpendicular to the outer circumferential surface.

Meanwhile, in a coupling relationship between the main body 111 and the electrode 112, the connection part 117 for transmitting and receiving a sensing value of the electrode 112 may electrically connect the electrode 112 and the controller 130 through the inside of the main body 111. More specifically, the main body 111 may be coupled so that sewage cannot be introduced into the main body 111 because there is no gap between the electrode part 112 and the connection part 117. For example, the sealing between the space inside the main body 111 and the external space may be maintained by using a rubber packing.

The sensor part 110 may include a pollution prevention part 114 capable of emitting a fluid toward an electrode of the electrode part 112.

More specifically, the pollution prevention part 114 may include a fluid passage 115, which is a passage through which a fluid may move, and a pump part 116 that pumps the fluid to provide a force to move the fluid through the fluid passage 115. Here, the pump part 116 may be a fluid pump, which is a well-known technology, and thus a detailed description thereof will be omitted.

The fluid passage 115 of the sensor part 110 may be connected to the pump part 116 at one end thereof so that high-pressure fluid provided from the pump part 116 may move. In particular, it may be understood that the fluid passage 115 may be disposed through the interior of the main body 111.

In addition, the other end of the fluid passage 115 of the sensor part 110 may be provided inside the protection part 113 and may be connected to the fluid passage 115 and may be connected to at least one hole disposed toward the electrode part 112. Accordingly, when the pump part 116 emits a high pressure fluid, it is understood that the electrode 112 of the sensor 110 may be cleaned by emitting the fluid into the hole through the fluid passage 115. It is understood that the fluid emitted here may be a gas, and that the liquid and the gas may be emitted together. More preferably, a fluid to be emitted, including a liquid, may be suitable. However, the present invention is not limited thereto.

Here, at least one hole disposed toward the electrode part 112 of the protection part 113 may be disposed in an inner direction perpendicular to the outer circumferential surface of the protection part 113, but is not limited thereto. For example, the hole of the protection part 113 may be disposed at an angle between an inner direction perpendicular to the outer circumferential surface of the protection part 113 and the longitudinal direction of the main body 111, and thus a fluid emitted through the hole of the protection part 113 may move a contaminant through a structure opened in the longitudinal direction of the main body 111.

The sensor part 110 may include a connection part 117 electrically connected to the electrode part 112 and penetrating the main body 111 to be electrically connected to the controller 130. It may be understood that the connection part 117 may include a line for supplying power to the electrode part 112 or a line for transmitting and receiving a value sensed through the electrode part 112.

The communication part 120 may communicate with the server 200 through the network 1 and transmit data obtained by processing the measured sensing value through the sensor 110 to the server 200. The communication part 120 may be configured as a wireless communication module of a known type.

The controller 130 may include a processor 131 and a memory 132.

The processor 131 may process at least one sensing value measured from the sensor part 110 and output a signal for controlling connection with the communication part 120, the connection part 117, and the connection unit 140. For example, the processor 131 may include a digital signal processor (DSP) and/or a micro control unit (MCU).

The processor 131 may measure at least one basic water quality measurement item, for example, turbidity, kind of turbidity, residual chlorine concentration, hydrogen ion concentration, temperature, electrical conductivity, dissolved oxygen amount, oxidation degree, etc measured from the sensor part 110, in a predetermined time unit, and process the measured sensing value. Accordingly, the processor 131 may generate a signal for controlling the communication part 120 to transmit the processed sensing value to the server 200.

In addition, the processor 131 may generate a signal for controlling the pollution prevention part 114. More specifically, the processor 131 may generate a signal for controlling the pollution prevention part 114 to emit a fluid to clean the sensor part 110.

For example, when the sensor part 110 is cleaned at each predetermined period, the processor 131 may output a more accurate sensing value, and thus may generate a signal for controlling the pollution prevention part 114 to emit the fluid at each predetermined period. Accordingly, the pump part 116 of the pollution prevention part 114 may emit the fluid based on the control signal generated by the processor 131.

In another embodiment, the processor 131 may generate various control signals for driving the pump part 116. For example, the processor 131 may generate a signal for controlling the pump part 116 to strongly spray the fluid for a short period, and may rapidly control the on/off to generate a signal for controlling the pump part 116 to spray the fluid together with vibration. However, the present invention is not limited thereto.

Meanwhile, in the case where the sensor part 110 includes a plurality of sensors, based on processing the plurality of sensing values received from the sensor part 110, the processor 131 may generate a signal for controlling the pollution prevention part 114 to emit the fluid when a change amount of at least one sensing value is identified during the predetermined period by using the processing of the plurality of sensing values. More specifically, the processor 131 may generate a signal for controlling the pump part 116 of the pollution prevention part 114 to drive.

When the sensor part 110 includes a plurality of sensors, the sensor part 110 may measure at least two sensing values among the basic water quality measurement items, and when one sensing value is changed significantly during the predetermined period, the processor 131 may control the pollution prevention part 114 to clean the sensor part 110 in order to secure reliability of whether the accurate sensing value is identified. However, the present invention is not limited thereto.

In another embodiment, when the sensor part 110 includes a plurality of sensors, when only one sensing value is changed significantly during the predetermined period and another sensing value is not changed, a problem such as a failure of a sensor outputting another sensing value due to a correlation between the basic water quality measurement items or a data reliability decrease due to a pollutant may be suspected. Accordingly, the processor 131 may generate a control signal for cleaning the sensor part 110 by controlling the pollution prevention part 114.

More specifically, when the sensor part 110 includes a first sensor and a second sensor, based on processing the sensing values received from the first sensor and the second sensor, the processor 131 may generate a signal for controlling the pollution prevention part 114 to emit the fluid when a change amount of at least one sensing value is identified during the predetermined period by using the processing of the plurality of sensing values. Here, the predetermined period and the predetermined value may be periods and values determined experimentally and empirically. For example, the case where the change amount of a specific value is sensed during the predetermined period may be a case where sewage having high reliability and a very high pollution degree is introduced among the entire acquisition data.

In another embodiment, where the sensor part 110 includes a first sensor and a second sensor, the processor 131 may identify, based on processing the sensing values received from the first sensor and the second sensor, an amount of change in the sensing value of the first sensor that is greater than a predetermined value over a predetermined period of time. If the sensing value of the second sensor does not change by a predetermined amount over a predetermined period, the processor 131 may determine with a high degree of confidence that contaminants in the second sensor are obscuring the sensor and preventing accurate sensing values from being identified. More specifically, the meaning of unchanging by a predetermined amount over a predetermined period may mean that a change amount greater than a first predetermined value is identified during a predetermined period of the first sensor and a sensing value of the second sensor is less than a second predetermined value during the predetermined period.

Accordingly, the processor 131 may generate a signal for controlling the pump part of the pollution prevention part included in the second sensor to clean the second sensor. Here, the predetermined period and the predetermined value may be periods and values determined experimentally and empirically. For example, the value may be determined based on a degree of correlation between the change amount of the sensing value of the first sensor and the change amount of the sensing value of the second sensor. However, the present invention is not limited thereto.

The memory 132 may store a program for performing the above-described operation and the following operation, and the processor 131 may execute the stored program. When the plurality of memories 142 and the processor 131 are provided, they may be integrated into one chip and may be provided in a physically separated position. The memory 142 may include a volatile memory such as a static random access memory (S-RAM), a dynamic random access memory (DRAM), or the like for temporarily storing data. In addition, the memory 142 may include a non-volatile memory such as a read only memory (ROM), an erasable programmable read only memory (EPROM), an electrically erasable programmable read only memory (EEPROM), or the like for storing a control program and control data for a long time. The processor 131 may include various logic circuits and arithmetic circuit, and may process data according to a program provided from the memory 142 and generate a control signal according to a result of the processing.

More specifically, the memory 132 may store data obtained by the sensor part 110 and/or the communication part 120.

The water quality of sewage measurement electronic device 100 may further include a connection unit 140 that may be detachably coupled to the connection part 117 of the sensor part 110. More specifically, the connection unit 140 may be a unit provided to be easily detachable because the type of the sensor needs to be changed according to the use of the water quality of sewage measurement electronic device 100. For example, the connection unit 140 may have a hole configured to be inserted into and coupled to a pin of the connection part 117.

Meanwhile, the water quality of sewage measurement electronic device 100 may include a display (not shown).

The display (not shown) may be provided on the front surface and/or the rear surface of the controller 130 to display a state of the water quality of sewage measurement electronic device 100 and/or sensing values of basic water quality measurement items measured by the sensor part 110. To this end, the display (not shown) may be provided as a display module of a known type. However, the present invention is not limited thereto. For example, it may be understood that the display (not shown) may be disposed at a position close to the housing and/or the controller 130 of the water quality of sewage measurement electronic device 100. In addition, the display (not shown) may be also used as an input device that is provided integrally with an input device capable of receiving a user input and inputs a user manipulation.

That is, the display (not shown) may be a device that transmits a user input to the controller 130 based on a user input (e.g., manipulation), and is not limited thereto.

Meanwhile, the display (not shown) according to an embodiment may include a cathode ray tube (CRT), a Flat Panel Display (FPD), a liquid crystal display (LCD), a plasma display panel (PDP), a light emitting diode (LED), and an organic light emitting diode (OLED), but is not limited thereto. Accordingly, a display type or type known in the art or to be developed in the future.

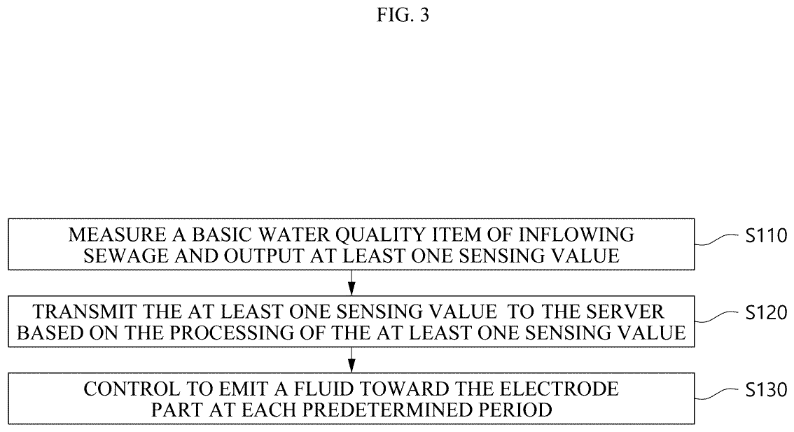

FIG. 3 is a flowchart illustrating a vehicle control mechanism of a driver assistance method according to an embodiment.

The water quality of sewage measurement method illustrated in FIG. 3 may be performed by the above-described water quality of sewage measurement electronic device 100. Accordingly, even though the contents omitted below, the contents described with respect to the water quality of sewage measurement electronic device 100 may be equally applied to the description of the water quality of sewage measurement method.

Referring to FIG. 3, the water quality of sewage measurement electronic device 100 may measure a basic water quality item of inflowing sewage and output at least one sensing value (S110).

In addition, the water quality of sewage measurement electronic device 100 may transmit the at least one sensing value to the server 200 based on the processing of the at least one sensing value output in operation S110.

In addition, the water quality of sewage measurement electronic device 100 may emit a fluid toward the electrode part 112 at each predetermined period (S130). More specifically, the controller 130 of the water quality of sewage measurement electronic device 100 may generate a signal for controlling the pollution prevention part 114 to emit a fluid toward the electrode part 112 at each predetermined period.

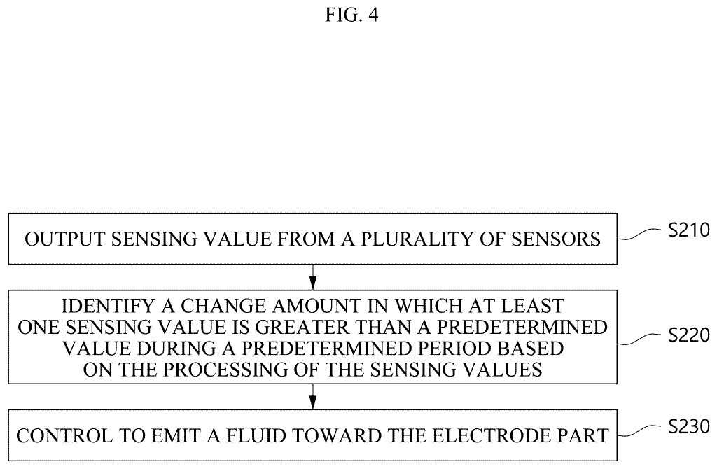

Referring to FIG. 4, the water quality of sewage measurement electronic device 100 may generate a signal for controlling the sensor part 110 including a plurality of sensors and output a plurality of sensing value (S210).

In addition, the water quality of sewage measurement electronic device 100 may identify a change amount in which at least one sensing value is greater than a predetermined value during a predetermined period based on the processing of the plurality of sensing values output in operation S210 (S220).

In addition, the water quality of sewage measurement electronic device 100 may emit a fluid toward the electrode part 112 in response to the identifying of the large change amount in operation S220. S230). More specifically, the controller 130 of the water quality of sewage measurement electronic device 100 may generate a signal for controlling the pollution prevention part 114 to emit a fluid toward the electrode part 112 in response to the identifying of the large change amount in operation S220.

The disclosed embodiments may be implemented in the form of a recording medium storing instructions executable by a computer. The instructions may be stored in the form of program code, and when executed by a processor, the instructions may generate a program module to perform operations of the disclosed embodiments. The recording medium may be implemented as a computer-readable recording medium.

The computer-readable recording medium includes all types of recording media storing instructions that can be read by a computer. For example, the storage device may include a read only memory (ROM), a random access memory (RAM), a magnetic tape, a magnetic disk, a flash memory, an optical data storage device, and the like.

The disclosed embodiments will be described with reference to the accompanying drawings as described above. Those of ordinary skill in the art will understand that the present invention may be practiced in a form different from the disclosed embodiments without changing the technical spirit or The disclosed embodiments are illustrative and should not be construed as limiting.

1. An electronic device for measuring water quality of inflow sewage comprising:
a sensor part installed inside a pipeline of the inflow sewage and including at least one sensor for measuring a water quality item of the inflow sewage and outputting a sensing value;
a communication part for communicating with a server; and
a controller for processing and transmitting the sensing value to the server,
wherein the sensor part further includes a pollution prevention part for emitting a fluid toward an electrode part of the sensor, and
wherein the controller controls the pollution prevention part to emit the fluid at a first predetermined period.

2. The electronic device of claim 1,
wherein the sensor comprises:
a main body extending in a longitudinal direction of a cylindrical shape;
at least one electrode part protruding in the longitudinal direction from the main body; and
a protection part,
wherein one end of the protection part is connected to an outer circumference of one end of the body and extends in the longitudinal direction to surround the protruding electrode part, and the other end is open to have a space provided therein.

3. The electronic device of claim 2,
wherein the pollution prevention part includes a fluid passage, wherein one end of the fluid passage is connected to a pump part so that a fluid moves through the main body, and
wherein the protection part includes at least one hole, wherein the at least one hole is provided toward the electrode part inside the protection part and connected to the fluid passage.

4. The electronic device of claim 2,
wherein at least a portion of an outer circumferential surface of the protection part is opened so that foreign substances removed by the pollution prevention part move.

5. The electronic device of claim 2,
wherein the sensor further comprises:
a connection part capable of transmitting and receiving the sensing value, and
wherein the electronic device further comprises:
a connection unit configured to be detachably coupled to the connection part.

6. The electronic device of claim 1,
wherein the sensor part includes a first sensor and a second sensor, and
wherein the controller controls the pollution prevention part to emit a fluid when a change amount of at least one sensing value greater than a predetermined value during a second predetermined period is identified based on processing sensing values of the first sensor and the second sensor.

7. The electronic device of claim 5,
wherein the sensor comprises at least one DO sensor, a pH sensor, an electrical conductivity sensor, a Mixed Liquor Suspended Solid (MLSS) sensor, a turbidity sensor or a residual chlorine sensor.

8. A method for measuring water quality of sewage using an electronic device for measuring water quality of inflow sewage, comprising:
measuring a water quality item of the inflow sewage by a sensor part including at least one sensor to output at least one sensing value;
processing the sensing value by a controller to transmit the processing to a server through a communication part; and
controlling a pollution prevention part to emit a fluid toward an electrode part of the sensor by a controller at a first predetermined period.

9. The method of claim 8,
wherein the sensor comprises:
a main body extending in a longitudinal direction of a cylindrical shape;
at least one electrode part protruding in the longitudinal direction from the main body; and
a protection part,
wherein one end of the protection part is connected to an outer circumference of one end of the body and extends in the longitudinal direction to surround the protruding electrode part, and the other end is open to have a space provided therein.

10. The method of claim 9,
wherein the pollution prevention part includes a fluid passage, wherein one end of the fluid passage is connected to a pump part so that a fluid moves through the main body, and
wherein the protection part includes at least one hole, wherein the at least one hole is provided toward the electrode part inside the protection part and connected to the fluid passage.

11. The method of claim 9,
wherein at least a portion of an outer circumferential surface of the protection part is opened so that foreign substances removed by the pollution prevention part move.

12. The method of claim 9,
wherein the sensor further comprises:
a connection part capable of transmitting and receiving the sensing value, and
wherein the electronic device further comprises:
a connection unit configured to be detachably coupled to the connection part.

13. The method of claim 8,
wherein the sensor part includes a first sensor and a second sensor, and
further comprising: controlling the pollution prevention part to emit a fluid when a change amount of at least one sensing value greater than a predetermined value during a second predetermined period is identified based on processing sensing values of the first sensor and the second sensor.

14. The method of claim 12,
wherein the sensor comprises at least one DO sensor, a pH sensor, an electrical conductivity sensor, a Mixed Liquor Suspended Solid (MLSS) sensor, a turbidity sensor or a residual chlorine sensor.

15. A computer-readable recording medium having recorded thereon a program capable of executing the method of measuring water quality of sewage of claim 8.
